(19) United States
(12) Patent Application Publication
(10) Pub. No.: US 2011/0201902 A1
(43) Pub. Date: Aug. 18, 2011

(54) HEALTH CONDITION DETERMINING DEVICE

(75) Inventors: Toshikazu SHIGA, Otsu-shi (JP); Yoshitake OSHIMA, Kyoto-shi (JP); Masakazu TSUTSUMI, Kyoto-shi (JP); Hiroshi NAKAJIMA, Kyoto-shi (JP); Naoki TSUCHIYA, Kyoto-shi (JP); Mitsuhiro YONEDA, Kyoto-shi (JP)

(73) Assignee: OMRON HEALTHCARE CO., LTD., KYOTO-SHI (JP)

(21) Appl. No.: 13/091,973

(22) Filed: Apr. 21, 2011

Related U.S. Application Data
(63) Continuation of application No. PCT/JP2009/068821, filed on Nov. 4, 2009.

(30) Foreign Application Priority Data
Nov. 19, 2008 (JP) 2008-295910

(51) Int. Cl. A61B 5/00

(52) U.S. Cl. 600/300

(57) ABSTRACT

A health condition determining device accumulates data measured or input by an evaluation subject for each index of a plurality of items including a lifestyle index, which is an index related to body activity or lifestyle habit, and a biomarker, which is an index related to physiological state of the body. The health condition determining device then selects one or a plurality of biomarkers that lowers the evaluation of the health condition of the evaluation subject as a problematic index, extracts one or a plurality of lifestyle indices having the highest correlation with the problematic index as an improvable factor by comparing accumulated past data of the lifestyle index and the biomarker, and displays the improvable factor along with the evaluation of the health condition of the evaluation subject.

### TECHNICAL FIELD

The present invention relates to a technique for assisting personal and active self-health management targeting mainly healthy person or person having potentiality for suffering from illness.

### BACKGROUND ART

With increase in interest in health in the recent years, people who manage the blood pressure, the weight, the consumed calories and the like daily, and who actively exercise such as jogging and walking are increasing. Conventionally, various types of measurement devices such as a sphygmomanometer, a blood glucose meter, a weight scale, a body composition monitor, and a thermometer are widespreadly used as a health related equipment for personal use and home use, and a passometer, an activity amount meter, and the like are provided as a device for assisting exercise, which are used as one type of health management tool. However, the information that can be obtained with such devices is merely a numerical value (numerical value on the spot at the measured time point), and how to use such numerical value in health management depends on the user.

In view of such situation, the inventors of the present invention thoroughly reviewed the way the health management is supposed to be for individuals and homes, and the element technology necessary therefor.

Most conventional systems aim to provide the necessary numerical value information (blood pressure value, blood glucose value, etc.) for illness management and diagnosis. However, the user to be the target of health management for individuals and homes is not only person with illness but also a great number of healthy person and person having potentiality for suffering from illness (state in which the person does not have onset but has symptoms appearing at someplace in the body). In the case of healthy person and person having potentiality for suffering from illness, the measurement value obtained by the measurement device is in the normal range and thus the health condition (illness risk degree) cannot be grasped only with such values. Furthermore, the user cannot definitely specify how to take caution and how to manage what specific numerical values at the stage the kind of illness the user may develop is not known. That is, various biomarkers such as blood pressure value, blood glucose value, weight, body composition, body temperature and the like can be measured even at home by using various types of measurement devices, but most of the user do not know how to use the individual measurement value to health management. Various types of measurement devices are expected to be widespreadly used and an environment in which many types of biomarkers are measured daily at homes is expected to be realized in the future, but as the number of raw data obtained by measurement and the like increases and excessive information are obtained, it becomes difficult for the general user to obtain the significant information, that is, information advantageous to his/her own health management.

The information the healthy person or the person having potentiality for suffering from illness desires to know is not the individual measurement value at a certain time point, but is the comprehensive evaluation such as how health he/she is compared to others, to what extent he/she is healthy if he/she is healthy, or how serious if he/she is not healthy, and the specific guideline such as what actions to take to maintain his/her condition shown by the evaluation or to improve his/her condition shown by the evaluation.

“Continuity” is desired from the standpoint that cannot be missed to assist the health management for individuals and homes. For maintaining the healthy status or reducing the onset or risk of onset of the illness, the habitual practices such as measuring and evaluating biomarkers daily and trying to perform exercise periodically are the most effective, and advantageous information can be provided as the longer term measurement values are accumulated. In order to realize such continuity, a mechanism for enhancing and maintaining the motivation of the user is necessary, and how to provide the user with convincing and reliable information in an easily understandable way is assumed to be one key in realizing such mechanism. In a different way of viewing, the measurement device for personal and home use has the significance of existence in that the user can casually measure and accumulate the biomarkers periodically and daily rather than measurement on the spot for one time. Therefore, the health management for individuals and homes is not satisfied unless continuity has possibility and added value.

Systems for evaluating health conditions of an individual and proposing a target for health improvement disclosed in patent documents 1 to 4 are known. However, in patent document 1, there is a risk an objective evaluation may be inhibited since the user himself/herself can set valid/invalid for the index to be evaluated, and control the output of the system. In the method of grasping the lifestyle habit of the user from the questionnaire result and determining the items to improve and the target as in patent document 2, the causal correlation between the lifestyle habit, the biomarker and the health condition cannot be objectively evaluated, and hence a reliable improvement proposal cannot be provided. Furthermore, even if the value of the biomarker such as the blood pressure value and the total cholesterol is presented for the improvement target as in patent document 3 (see FIG. 10) and patent document 4 (see FIG. 8), the user cannot understand what kind of action to specifically take to achieve the target since this type of index cannot be freely controlled by the will of the user.
Patent Document 1: Japanese Unexamined Patent Publication No. 2006-65752
Patent Document 2: Japanese Unexamined Patent Publication No. 2006-119985
Patent Document 3: Japanese Unexamined Patent Publication No. 2006-163932
Patent Document 4: Japanese Unexamined Patent Publication No. 2007-122182

### DISCLOSURE OF THE INVENTION

### Problems to be Solved by the Invention

FIG. 14 shows a concept model of a health management system contrived by the inventors of the present invention. The system roughly has functions of three categories “CHECK”, “PLAN”, and “ACTION”, and comprehensively supports the cycle (hereinafter referred to as CPA cycle) of collecting information from a living body (CHECK), making a plan to maintain and improve health condition based on the information (PLAN), and assisting the implementation of the plan (ACTION). The continuity implementation of active self health management for individuals and homes is expected to be realized by providing such CPA cycle.

It is an object of the present invention to provide an element technology related to the PLAN function of the concept model. Specifically, one of the objects of the present invention is to provide a technology for presenting to the user a specific improvement action that is effective in improving the health condition of the user and that can be implemented by the will of the user. Another object of the present invention is to provide a technology for visualizing how the improvement in body activity and lifestyle habit in daily life influences the comprehensive health condition. Another further object of the present invention is to provide a technology for assisting the setting of an appropriate improvement target that can be realized at a reasonable level for the user.

### Means for Solving the Problem

To achieve the above object, the present invention adopts the following configuration. In other words, a health condition determining device according to the present invention includes a storage means for accumulating data measured or input from an evaluation subject for each index of a plurality of items including a lifestyle index, which is an index related to body activity or lifestyle habit, and a biomarker, which is an index related to physiological state of the body; an evaluating means for evaluating a health condition of the evaluation subject based on a plurality of indices including the biomarker; a problematic index selecting means for selecting one or a plurality of biomarkers that lowers the evaluation of the health condition of the evaluation subject from the biomarkers used in the evaluation of the evaluating means as a problematic index; an improvable factor extracting means for extracting one or a plurality of lifestyle indices having the highest correlation with the selected problematic index as an improvable factor by comparing past data of the lifestyle index and the biomarker accumulated in the storage means; and a display means for displaying the improvable factor along with the evaluation of the health condition of the evaluation subject.

In the present invention, various types of indices that may directly or indirectly influence the health of a person obtained by measurement or input are classified into “biomarker” and “lifestyle index” according to the respective characteristics. The biomarker indicates the physiological state of the body, and is useful as information for objectively and quantitatively evaluating the health condition of the person. In the present invention, the biomarker is mainly used to evaluate the health condition to obtain a highly reliable evaluation result. The biomarker (problematic index) which is a factor lowering the evaluation can also be extracted with high validity.

However, as the biomarker itself represents the physiological state of the body, it is difficult for a person to freely control it according to his/her own will. Thus, it is not appropriate to present the improvement of the biomarker such as “lower the blood pressure value to xx” as an advice for improving health. The evaluation subject (user) does not know what specific action to take to realize the presented target value.

In the present invention, the lifestyle index having the highest correlation with the problematic index is selected as the improvable factor, and such improvable factor is presented to the user. The lifestyle index, that is, the body activity and the lifestyle habit can be basically controlled according to his/her will, and thus the user can easily associate the same with a specific improvement activity. Furthermore, in the present invention, the lifestyle index effective for the improvement of the problematic index and the improvement of the health condition can be extracted since the correlation (causal relationship) between the lifestyle index and the biomarker is evaluated using the past data of the user himself/herself accumulated in the storage means.

In the present invention, an improvement target setting means for causing the evaluation subject to set an improvement target with respect to the improvable factor; and an improvement effect calculation means for predicting the improvement effect in the problematic index when the improvement target is achieved after the improvement target is set by the improvement target setting means are further preferably arranged, wherein the evaluating means preferably evaluates the health condition after the improvement by taking the improvement effect of the problematic index into consideration, and the display means further preferably displays the improvement target of the improvable factor and the evaluation of the health condition after the improvement.

According to such configuration, how the improvement in body activity and the lifestyle habit in daily life influence the comprehensive health condition can be visualized and simulated. The highly convincing information thus can be provided to the user, and the motivation with respect to health improvement can be maintained and enhanced.

The improvement target setting means preferably sets an upper limit of a value that can be set as the improvement target based on a distribution of values of the improvable factors accumulated in the storage means.

Thus, the setting of the appropriate improvement target that can be realized in a reasonable level for the user can be assisted by determining the upper limit based on the distribution (fluctuation) of the values of the improvable factors of the user himself/herself. The user himself/herself can also grasp the realistic improvement amount of the health condition at the current time point.

The improvement effect calculation means preferably models influence of the value of the improvable factor and change thereof on the value of the problematic index based on the past data of the improvable factor and the problematic index accumulated in the storage means, and calculates the improvement effect of the problematic index using the model.

The reliability in predicting the improvement effect thus can be enhanced by using the past data of the user himself/herself. However, if the accumulation of past data stored in the storage means, is small (does not satisfy a predetermined amount) such as at the beginning of use of the device, the model may be generated using the data (e.g., average data of a person of the same age and same gender) prepared in advance in the storage means.

The display means further preferably displays the evaluation of the health condition and an average value of the improvable factor in a person of the same age and same gender as the evaluation subject.

The user can intuitively grasp whether good or bad his/her health condition is and whether good or bad his/her lifestyle habit is by comparing with the average value. The effects for maintaining and enhancing the motivation of the user can be expected by displaying the comparison target.

If there are plural improvable factors, the display means further preferably displays intensity of the influence of each improvable factor on the problematic index.

Therefore, the user can grasp the improvement effect that can be expected on each improvable factor, so that the user can easily set the optimum improvement target that conforms to his/her lifestyle habit in view of the easiness in working on the improvement and the improvement effect that can be expected.

The present invention may provide a health condition determining device including at least some of the means described above, or a health condition determining system including the health condition determining device and one or more measurement devices. The present invention may also provide a health condition determining method including at least one part of the processes described above, a program for causing a computer to execute the method, or a recording medium recorded with such program. Each of the means and processes described above may be combined as much as possible to configure the present invention.

### Effect of the Invention

According to the present invention, a specific improvement activity that is effective in improving the health condition of the user and that can be implemented by the will of the user can be presented to the user. According to the present invention, how the improvement of the body activity and the lifestyle habit in daily life influences the comprehensive health condition can be visualized. Furthermore, according to the present invention, the setting of the appropriate improvement target that can be realized at a reasonable level of the user can be assisted.

### BRIEF DESCRIPTION OF THE DRAWINGS

FIG. 1 is a view showing an overall configuration of a health management system according to the present invention.

FIG. 2 is a view showing an influence propagation model between indices adopted in the present invention.

FIG. 3 is a view showing one configuration example of a comprehensive health condition determining system according to an embodiment of the present invention.

FIG. 4 is a block diagram schematically showing the hardware configuration of a comprehensive health condition determining device.

FIG. 5 is a function configuration diagram schematically showing the functions of the comprehensive health condition determining device.

FIG. 6 is a view showing a specific example of the influence propagation model.

FIG. 7 is a view showing one example of the screen display.

FIG. 8 is a flowchart showing the extracting process of the improvable factor.

FIG. 9 is a flowchart showing the details of the extracting process of the improvable factor.

FIG. 10 is a view showing another display example of the improvable factor.

FIG. 11 is a view schematically showing the method of modeling the influence of the change in a certain lifestyle index I on the value of the biomarker J.

FIG. 12 is a view schematically showing the method of obtaining the improvement effect from the model of FIG. 11.

FIG. 13 is a view schematically showing the method of predicting the improvement effect when the improvement target is set to a plurality of lifestyle indices.

FIG. 14 is a view showing a concept model of a health management system.

### BEST MODE FOR CARRYING OUT THE INVENTION

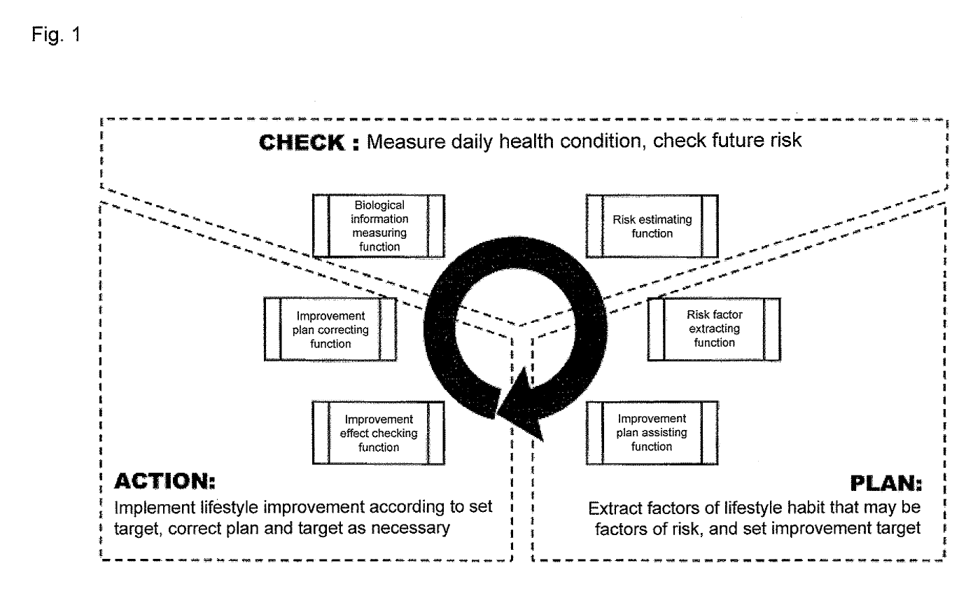

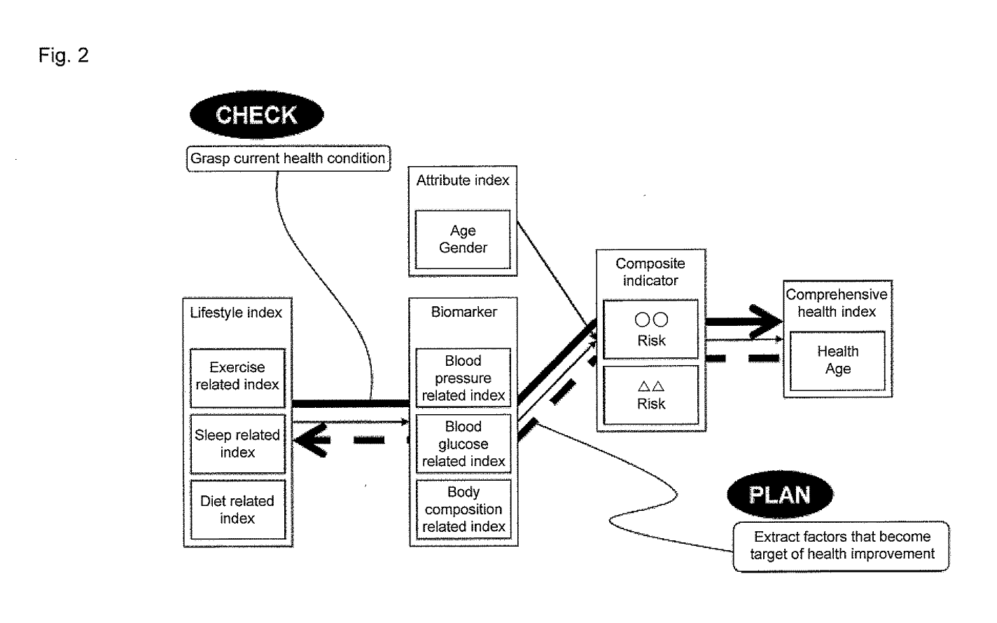

Preferred embodiments of the present invention will be illustratively described in detail below with reference to the drawings. First, the outline of the present invention will be described with reference to FIG. 1 and FIG. 2, and then the specific embodiments will be described.

(Outline of Health Management System)

FIG. 1 shows an overall configuration of a health management system according to the present invention. The health management system is a system for supporting the CPA cycle described above. The function related to “CHECK” includes “biological information measuring function” for measuring the daily health condition and “risk estimating function” for estimating the future risk and evaluating the comprehensive health condition from the information obtained by measurement. The function related to “PLAN” includes “risk factor extracting function” for extracting the factors that become the factors of risk based on the result obtained in CHECK, and “improvement plan assisting function” for setting the improvement target and proposing the improvement plan. The function related to “ACTION” includes “improvement effect checking function” for assisting the implementation of the lifestyle improvement activity (exercise) according to the improvement target and plan obtained in PLAN, and “improvement plan correcting function” for correcting the plan and the target as necessary.

With each function of CHECK, PLAN, and ACTION organically bonding and repeating the cycle, the determination of the comprehensive health condition based on a plurality of biomarkers, the evaluation of the future health risk, and the relationship between the risk and the activity in daily life can be visualized, and assisting of the continuous implementation of the active self health management for individuals and homes can be expected.

(Index and Influence Propagation Model)

FIG. 2 is a view showing an influence propagation model between indices adopted in the present invention. In the present invention, various types of indices that are obtained by measurement or input and that may directly or indirectly influence the health of a person are classified into three categories “attribute index”, “lifestyle index”, and “biological index” according to the characteristics. The hierarchical structure (causal structure) between the categories is determined in view of the propagation of influence between the indices. Furthermore, a plurality of types of “composite indicator” is introduced as an index representing the health condition of the person, and “comprehensive health index” obtained by comprehensively evaluating the composite indicators is also introduced.

The “attribute index” is the criterion for objectively identifying the attribute of an individual and the numerical value thereof, and includes gender, age, height, clinical history, and the like. The attribute index basically cannot be controlled by his/her will. The “lifestyle index” is the criterion indicating the daily body activity and lifestyle habit (exercise, sleep, diet, etc.) and the numerical value thereof. The lifestyle index includes exercise related index (intermittent walking time per unit time, continuous walking time, number of continuous walking, regularity of walking pattern, etc.) sleep related index (sleeping time, number of times of roll-over, number of times of breathing, etc.), diet related index (consumed amount of calorie, time of dinner, alcohol ingestion frequency, etc.), and other information (smoking habit or not, etc.). The lifestyle index basically can be controlled by his/her will, and is the target of control (improvable factor) for maintaining and improving health. The “biological index” is the criterion indicating the physiological state of the body and the numerical value thereof. The biological index includes blood pressure related index (systolic blood pressure, diastolic blood pressure), blood glucose related index (blood glucose value at time of empty stomach, as needed blood glucose value), body composition related index (weight, body fat percentage, muscle rate), and serum total cholesterol and the like. The biological index is useful as information for objectively and quantitatively showing the health condition of the person. However, the biological index is difficult to be controlled by his/her will, and is indirectly influenced by the control of the lifestyle index.

The “composite indicator” is a criterion indicating the risk with respect to a specific illness or the state of a specific body organ or body function based on the relationship of a plurality of indices selected from the biological index, the lifestyle index, and the attribute index and the statistically obtained epidemiological information (death rate etc.), and the numerical value thereof. The composite indicator indicating the risk on a specific illness includes apoplexy risk, cardiovascular related illness risk, coronary arterial abnormality risk, and the like. The composite indicator indicating the state of a specific body organ or body function includes blood vessel age, blood pressure age, body strength age, muscle age, and the like. The composite indicators are difficult to be directly controlled, and are indirectly influenced by the control of the lifestyle index. The “comprehensive health index” is the criterion indicating the relative health condition of an individual in a parent population based on the correspondence of the plurality of composite indicators and the tendency of the actual age of the large scale parent population, and the numerical value thereof. The comprehensive health index includes the health age, the health deviation value, and the like.

When evaluating the health condition with the CHECK function (risk estimating function) of FIG. 1, the composite indicator is first calculated from the measurement value and the like of the biomarker along the influence propagation model, and the comprehensive health index is obtained from the calculation result of the composite indicator. In the PLAN function (risk factor extracting function), the biomarker that is the factor for lowering the comprehensive health index and the composite indicator is specified by tracking the influence propagation model in the opposite direction, and the effective lifestyle index for improving the relevant biomarker can be extracted. When the improvement target of the lifestyle index is given in the PLAN function (improvement plan assisting function), the improvement effect of each of the biomarker, the composite indicator, and the comprehensive health index can be predicted by evaluating the propagation of the influence by the change in the value of the lifestyle index in order along the influence propagation model. Therefore, in the present invention, the health condition can be objectively and quantitatively evaluated, and how the body activity and the lifestyle habit in the daily life influence the health condition can be visualized by adopting the influence propagation model as in FIG. 2.

(Comprehensive Health Condition Determining System)

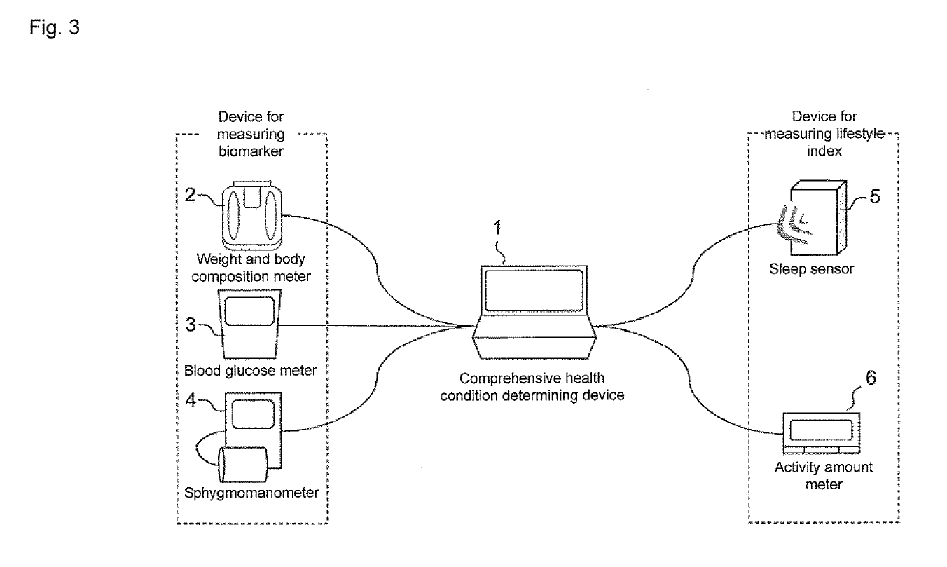

One specific embodiment of the present invention will now be described. FIG. 3 is a view showing one configuration example of a comprehensive health condition determining system (hereinafter also simply referred to as “system”) according to the embodiment of the present invention. The system is positioned as an element technology responsible for the CHECK function and the PLAN function of the configuration of the health management system shown in FIG. 1.

The system is configured by a comprehensive health condition determining device 1 and one or more measurement devices 2 to 6. The measurement device may be a device for measuring a biomarker from the body of a person, a device for measuring the lifestyle index such as the body activity and the lifestyle habit of the person, and the like. The measurement device of the biomarker includes a weight and body composition meter capable of measuring the weight, the body composition (body fat, muscle, etc.), BMI and the like, a blood glucose meter for measuring the blood glucose value, the sphygmomanometer for measuring the blood pressure and the pulse rate, the thermometer for measuring the body temperature, and the heart rate meter for measuring the heart rate. The measurement device of the lifestyle index includes the activity amount meter for measuring the body activity amount and the exercise intensity, the passometer for measuring the number of steps, the sleep sensor for measuring the state of sleeping, and the calorie meter for carrying out calorie calculation of the diet. In the system of the present embodiment shown in FIG. 3, the weight and body composition meter (2), the blood glucose meter (3), the sphygmomanometer (4), the sleep sensor (5) and the activity amount meter (6) are used.

The comprehensive health condition determining device 1 and each measurement device 2 to 6 are data communicable by wire or wirelessly. The measurement value obtained by each measurement device is sent to and aggregated in the comprehensive health condition determining device. If the comprehensive health condition determining device and the measurement device are connected on a steady basis, the data is transmitted from the measurement device to the comprehensive health condition determining device every time the measurement is carried out or at every timing determined in advance. The data between the devices are thereby synchronized. If the comprehensive health condition determining device and the measurement device are not connected on a steady basis, the measurement device or the comprehensive health condition determining device monitors the presence of connection, and the data is automatically synchronized when connection is detected. The measurement value may, obviously, be transferred to the health condition determining device by the operation of the user himself/herself.

(Hardware Configuration of Comprehensive Health Condition Determining Device)

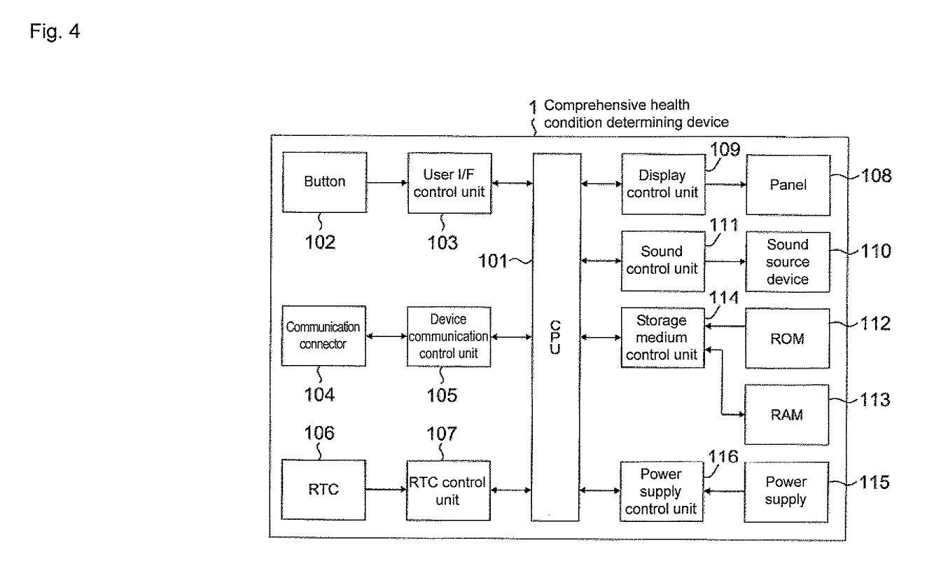

FIG. 4 is a block diagram schematically showing the hardware configuration of the comprehensive health condition determining device 1.

As shown in FIG. 4, the comprehensive health condition determining device 1 includes a CPU (Central Processing Unit) 101, a button 102 and a user I/F (interface) control unit 103, a communication connector 104 and a device communication control unit 105, an RTC (Real Time Clock) 106 and a RTC control unit 107, a panel 108 and a display control unit 109, a sound source device 110 and a sound control unit 111, a ROM (Read Only Memory) 112/RAM (Random Access Memory) 113 and a storage medium control unit 114, a power supply 115 and a power supply control unit 116. The device may be configured as a dedicated device, or may be configured by mounting the hardware (e.g., communication connector with measurement device) necessary for the versatile device such as the personal computer and the necessary program.

The button 102 is an input means for inputting information and instructions to the comprehensive health condition determining device 1. The information and instructions input by the operation of the button 102 are notified to the CPU 101 through the user I/F control unit 103.

The communication connector 104 and the device communication control unit 105 are communication means for realizing data communication between various types of measurement devices. The communication method may be a wired communication including USB and IEEE1394, or a wireless communication including Bluetooth, ZigBee, IrDA, and wireless LAN.

The RTC 106 and the RTC control unit 107 are portions that provide the timing function.

The panel 108 and the display control unit 109 are display means for displaying various types of indices, to be described later. A liquid crystal display, an organic EL display, and the like are suitably used for the panel 108.

The sound source device 110 and the sound control unit 111 are output means for outputting alert, audio guidance, and the like.

The ROM 112 is a storage medium for storing programs that provide functions of the comprehensive health condition determining device 1, various types of set values, measurement values acquired from each measurement device 2 to 6, information input from the input means, various types of indices to be described later, and the like. The ROM 112 is configured with a rewritable memory such as the EEPROM (Erasable Programmable ROM). The RAM 113 is a storage medium used as a work memory when executing the program. The access to the ROM 112 and the RAM 113 is controlled by the storage medium control unit 114. The storage medium such as the hard disc may be arranged in addition to the EEPROM or in place of the EEPROM.

The power supply 115 and the power supply control unit 116 are functions for supplying power to the comprehensive health condition determining device 1. The power supply 115 may be a battery or an AC power supply.

(Function Configuration of Comprehensive Health Condition Determining Device)

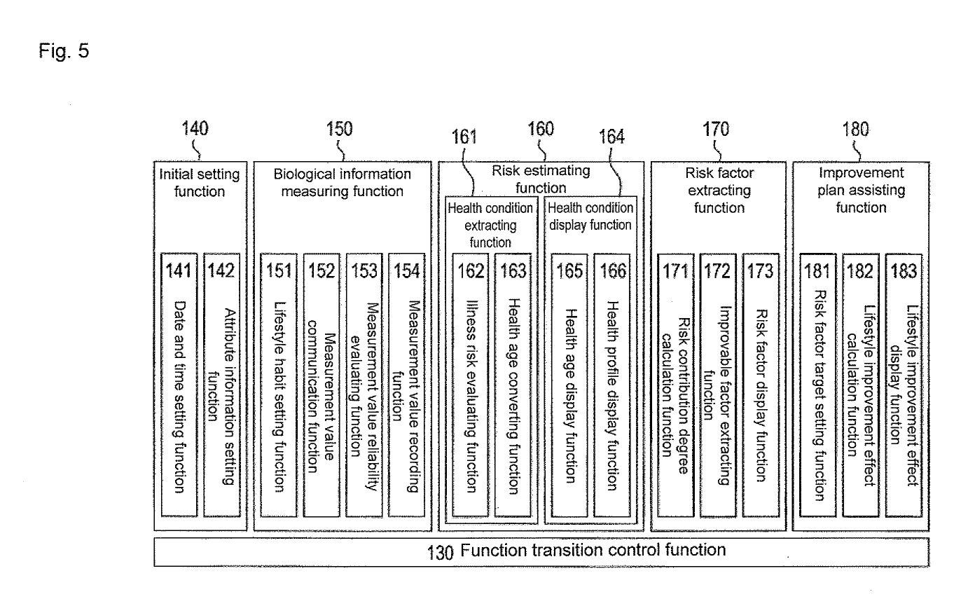

FIG. 5 is a function configuration diagram schematically showing the functions of the comprehensive health condition determining device 1.

As shown in FIG. 5, the comprehensive health condition determining device 1 includes, for the functions thereof, a function transition control function 130, an initial setting function 140, a biological information measuring function 150, a risk estimating function 160, a risk factor extracting function 170, and an improvement plan assisting function 180. The initial setting function 140 includes a date and time setting function 141 and an attribute information setting function 142. The biological information measuring function 150 includes a lifestyle habit setting function 151, a measurement value communication function 152, a measurement value reliability evaluating function 153, and a measurement value recording function 154. The risk estimating function 160 includes a health condition extracting function 161 and a health condition display function 164, in which the health condition extracting function 161 includes an illness risk evaluating function 162 and a health age converting function 163, and the health condition display function 164 includes a health age display function 165 and a health profile display function 166. The risk factor extracting function 170 includes a risk contribution degree calculation function 171, an improvable factor extracting function 172, and a risk factor display function 173. The improvement plan assisting function 180 includes a risk factor target setting function 181, a lifestyle improvement effect calculation function 182, and a lifestyle improvement effect display function 183. The functions are realized by having the CPU 101 read and execute the program stored in the ROM 112.

(Function Transition Control Function)

The function transition control function 130 is a function for comprehensively controlling the switching of the initial setting function 140, the biological information measuring function 150, the risk estimating function 160, the risk factor extracting function 170, and the improvement plan assisting function 180 according to the operation of the user, the communication with the measurement device, the event (interruption) that occurred in the program being executed, and the like.

(Initial Setting Function)

The date and time setting function 141 is a function for setting the current date and time (local time) in the device. In the date and time setting function, a setting screen such as “year/month/day?______” is displayed on the panel 108, and the user is urged to set the current date and time. When the current date and time is set by the operation of the input means such as the button 102, the value is written to the RTC 106. Thereafter, the RTC 106 times the current date and time.

The attribute information setting function 142 is a function for causing the user to input the attribute index. The “age” and “gender” are input for the attribute index. In the attribute information setting function 142, an input screen of the attribute index such as “age?______” is displayed on the panel, and the user is urged to input the attribute index. When the attribute index is input by the operation of the input means such as the button, the information is recorded in the user information DB in the ROM 112.

(Biological Information Measuring Function)

The lifestyle habit setting function 151 is a function for causing the user to input the lifestyle index. The “smoking habit” is input for the lifestyle index. In the lifestyle habit setting function 151, an input screen such as “smoking habit ? YES/NO” is displayed on the panel 108, and the user is urged to input. When the lifestyle index is input by the operation of the input means such as the button 102, the information is recorded in the user information DB in the ROM 112.

The measurement value communication function 152 is a function for acquiring the measurement value from various types of measurement devices through the communication connector 104. The acquired measurement value is accumulated in the ROM 112 by the measurement value recording function 154. In this case, the measurement value is recorded along with the time stamp representing the measurement date and time or the acquired date and time of the measurement value. The “systolic blood pressure value” acquired from the sphygmomanometer, the “blood glucose value” acquired from the blood glucose meter, the “weight” and the “body fat percentage” acquired from the weight and body composition meter are respectively recorded for the biomarker. Furthermore, the “sleeping time” and the “sleeping depth” acquired from the sleep sensor, the “intermittent walking time”, “continuous walking time”, “number of continuous walking”, and “walking pattern variation” acquired from the activity amount meter are respectively recorded for the lifestyle index. The continuous walking time is the cumulative value per one day of the time continuously walked for a predetermined time or longer, and the intermittent walking time is the value obtained by subtracting the continuous walking time from the total walking time of one day. The number of continuous walking is the number of times the continuous walking is carried out in one day.

The measurement value reliability evaluating function 153 is a function for evaluating the reliability of the measurement value. The reliability of the measurement value is the information (criterion) representing the extent of variation anticipated in the measurement value. It can also be considered as the uncertainty (or certainty) of the measurement value. The reliability of the measurement value is influenced by the accuracy of the measurement device, the measuring environment, the measurement mode, and other disturbances. For instance, the accuracy may possibly differ between the wrist type and the upper arm type even if the sphygmomanometer is the same, and the accuracy may possibly differ between the high end device and the wide spread device, Furthermore, the measurement value may vary even if the same device is used due to the influence of the place to carry out the measurement, the outside temperature, and the like. The measurement mode is, for example, the prediction mode and the actual measurement mode of an electronic thermometer. The accuracy of the prediction mode is inferior to that of the actual measurement mode. The measurement value may also vary due to the position of measurement and other disturbance factors. If the accuracy of the measurement device can be acquired from the measurement device, the measurement value reliability evaluating function 153 may use it for the reliability of the measurement value. Moreover, the measurement value also varies depending on the state (physical condition, fatigue, sleep, diet, etc.) of the person being measured, and hence the measurement reliability evaluating function 153 can evaluate the distribution and the variation of the measurement value of a predetermined period in the past, and use the same as the reliability of the measurement value.

Various methods of expressing the information representing the reliability of the measurement value can be assumed, and all such methods may be adopted. For instance, the reliability may be expressed by the range of variation such as “systolic blood pressure: 135±6”, or the reliability may be expressed by the range of value such as “systolic blood pressure value: 128 to 139”. Furthermore, the distribution and variation of the measurement value may be expressed with an average value and dispersion. Alternatively, the range of variation and the boundary of the range of value may be made indistinct by the fuzzy set such as “systolic blood pressure value: 135±about 6” and “systolic blood pressure value: about 128 to about 139”. The expression “the value has a width” is used to mean that “the value contains the information representing the reliability, and is not defined by one definite numerical value”.

The measurement values are collected as needed from the various types of measurement devices by the biological information measuring function 150 described above, and accumulated in the user information DB in the ROM 112 along with the information representing the reliability of the measurement value as necessary. In other words, the user information DB in the ROM 112 corresponds to the storage means for storing data measured or input from the evaluation subject with respect to each index of a plurality of items in the present invention.

(Risk Estimating Function: Health Condition Extracting Function)

The health condition extracting function 161 is configured by the illness risk evaluating function 162 and the health age converting function 163. In the present embodiment, the health condition extracting function 161 corresponds to the evaluating means for evaluating the health condition (illness risk and health age) of the evaluation subject based on a plurality of indices including the biomarker in the present invention.

((Illness Risk Evaluating Function))

The illness risk evaluating function 162 is a function for estimating the illness risk or the composite indicator using the biomarker, the lifestyle index, and the attribute index accumulated in the user information DB (information accumulated in the user information DB are hereinafter referred to as original index).

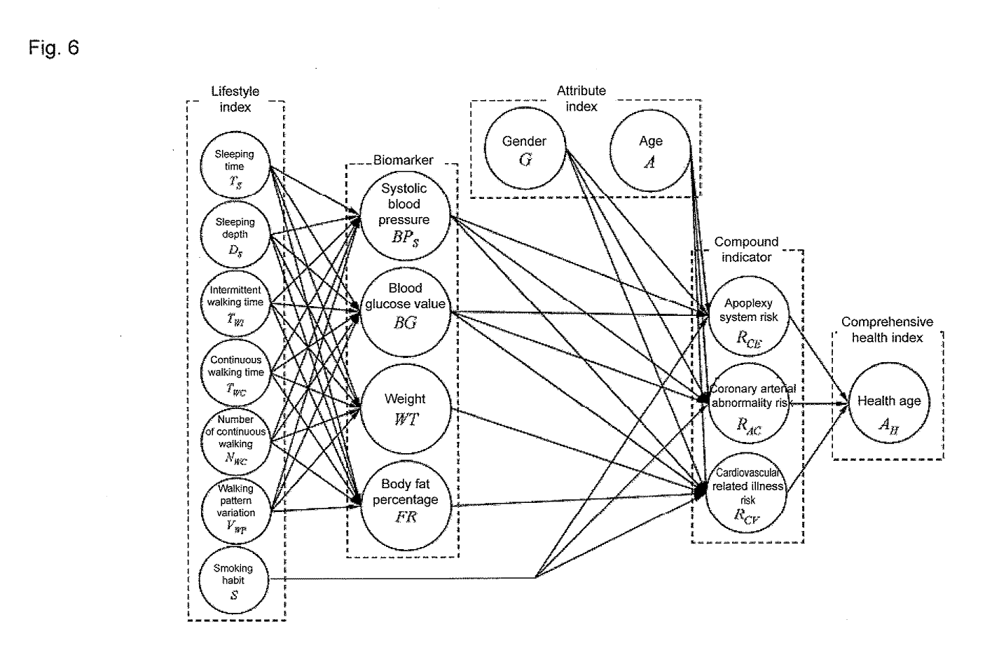

FIG. 6 shows a specific example of the influence propagation model in the present embodiment. Here, “gender G” and “age A” are used for the attribute index, and “systolic blood pressure value BPs”, “blood glucose value BG”, “weight WT”, and “body fat percentage FR” are used for the biomarker. The “sleeping time Tₛ”, “sleeping depth Dₛ”, “intermittent walking time T_{w1}”, “continuous walking time T_{wc}”, “number of continuous walking N_{wc}”, “walking pattern variation V_{wp}”, and “Smoking habit S” are used for the lifestyle index. Although not illustrated, “alcohol ingestion frequency”, “dinner time”, and the like are also used as the lifestyle index. The “apoplexy risk R_{CE}”, “coronary arterial abnormality risk R_{AC}” and “cardiovascular related illness risk R_{CV}” are used for the composite indicator. These indices are examples, and lesser number or greater number of indices may be used. The risk related to other illnesses may be used, and indices related to body organs and body functions such as the blood vessel age and the muscle age may be used for the composite indicator. The causal relationship between the original index and the composite indicator may be designed by a person from the knowledge of epidemiology, or may be automatically generated using a known causal structure estimating method.

As shown in FIG. 6, the illness risk evaluating function obtains each illness risk from seven indices (influencing factors) of gender G, age A, systolic blood pressure value BPₛ, blood glucose value BG, weight WT, body fat percentage FR, and smoking habit. In the present embodiment, a proportional hazard model is used as the risk evaluating model for calculating the illness risk. In other words, the risk R_{K} of the illness K at a certain time point t is expressed with the product of R_{K}(t) or the function of time and the exponential function of the linear sum of each influencing factor xⱼ, as shown in equation (1).

### Model of Illness Risk:

R_{K}=R_{K}(t)×exp(Σα_{jK}xⱼ+ε_{K}) (1)

Here, R_{K}(t) is the statistical death rate after time t by the illness K, and is referred to as the reference hazard. For instance, if the original parameter is 100 persons, all 100 persons are alive at the time point of t=0, and hence R(0)=0%. If nine persons died of illness K and ten persons cannot be confirmed whether alive or not due to death from other causes, immigration, and the like, the parameter is assumed as 90 persons (=100−10 persons) and R_{K}(t1)=( 9/90)×100%=10%. Therefore, the pure death rate due to the illness K can be expressed by excluding the number of fatalities (including person whose existence cannot be confirmed) due to causes other than the illness K from the parameter with elapse of time. The death rate is selected for the reference hazard R_{K}(t) herein, but the rate of occurrence of event other than death may be selected for the reference hazard R_{K}(t). For instance, the rate of occurrence of event that may affect the daily life such as hospitalization due to illness K, disorder, sever symptoms, and the like may be selected.

α_{jK} is a parameter (weight) showing the influence intensity of each factor xⱼ on the R_{K}(t). ε_{K} is a parameter showing the influence on the R_{K}(t) by factors other than the factor xⱼ. The method of generating the parameters α_{jK}, ε_{K} may be the Exact method (Monte Carlo accuracy probability test), Breslow method, Efron method (boot strap), discrete method, or the like.

In the present embodiment, the epidemiological data or the accomplishment of the large scale epidemiology research is used for the basic data for calculating the reference hazard R_{K}(t) and the parameters α_{jK}, ε_{K} of the risk evaluating model. The representative epidemiology research includes NIPPON DATA 80, Framingham study, Osaka research, Hisayamacho research, Suita research and the like. High accuracy can be realized in the risk evaluating model by obtaining the influence intensity between the factors and the death rate at each time point based on such epidemiological data. Furthermore, the understanding and the reliability of the user with respect to the evaluation index output by the present system can be expected to enhance and the motivation of the health management can be expected to enhance by using the epidemiological data as evidence.

((Health Age Converting Function))

The health age converting function 163 is a function for comprehensively evaluating a plurality of illness risks obtained by the illness risk evaluating function 162, and calculating the “health age”, which is an index in which the health condition of the user is converted to age.

In the present embodiment, the health age A_{H} is calculated using the linear sum model of the apoplexy risk R_{CE}, the coronary arterial abnormality risk R_{AC} and the cardiovascular related illness risk R_{CV}. βn is a parameter showing the influence intensity of each illness risk on the health age. ε is a parameter showing the influence on the health age by factors other than R_{CE}, R_{AC} and R_{CV}. The parameters βn, ε can be calculated through methods such as regression analysis using the epidemiological data described above. If the value of the illness risk includes information on the reliability such as the range of value and the distribution, the value of the health age A_{H} also has width.

### Model of Health Age:

A_{H}=β₁R_{CE}+β₂R_{AC}+β₃R_{CV}+ε (2)

(Risk Estimating Function: Health Condition Display Function)

The health condition display function 164 is a function for displaying the various types of indices obtained with the risk estimating function described above on the panel 108, and is configured by the health age display function 165 and the health profile display function 166. The health age display function 165 is a function responsible for the display of the “health age” or the comprehensive health index and the “illness risk” or the composite indicator, and the health profile display function 166 is a function for displaying to what extent each factor (mainly biomarker) used in the calculation of the illness risk influences the illness risk and the health age, that is, the influence intensity of each factor.

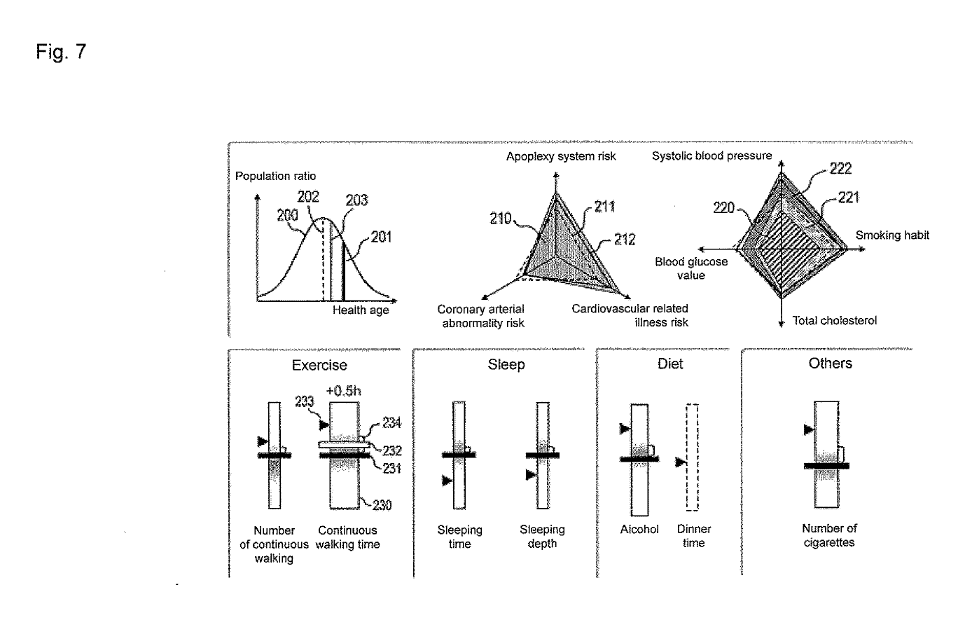

FIG. 7 shows one example of the screen display. The left side in the upper level of the screen is the display example of the “health age”. In this example, the health age 201 of the user is shown overlapping the health age distribution 200 (horizontal axis: health age, vertical axis: population ratio) in the set of the same age and the same gender as the user. As a comparative target, an average value 202 of the same age is also displayed. The health age distribution 200 and the average value 202 used are those calculated in advance for every age and every gender based on the epidemiological data. The user can intuitively recognize to what extent his/her health condition is better (worse) compared to the average of the person of the same age by looking at the display. The health age is also preferably displayed with a numerical value. The user can then intuitively understand whether good or bad his/her health condition is through comparison of the health age and his/her actual age. The health age may be preferably displayed not as a definite value but as a value having width according to reliability (uncertainness).

The middle in the upper level of the screen of FIG. 7 is the display example of the “illness risk”. In this example, the risk distribution 210 of three illness risks or the basis of calculation of the health age is displayed using the radar chart. Each axis of the radar chart is normalized with the value of the average illness risk of the person of the same age and same gender as the user, and the distribution 211 of the average illness risk is also displayed as the comparison target. The user can easily grasp to what extent his/her illness risk is diverged from the average and what risk factor is affecting his/her health age the greatest.

The right side in the upper level of the screen of FIG. 7 is the display example of the “health profile” by the health profile display function 166. In this example, the respective influence intensity of the four biomarkers (systolic blood pressure value BPₛ, blood glucose value BG, weight WT, and body fat percentage FR) commonly used in the calculation of each illness risk are shown using the radar chart. The influence intensity distribution 220 with respect to the coronary arterial abnormality risk, the influence intensity distribution 221 with respect to the cardiovascular related illness risk, and the influence intensity distribution 222 with respect to the apoplexy risk are cumulated in order from the center of the radar chart. That is, the outermost part shows the influence intensity distribution of each biomarker with respect to the health age. The user can easily grasp which biomarker should be paid attention in managing and improving his/her health by looking at the health profile. The influence intensity with respect to the illness risk is calculated by the risk contribution degree calculation function 171 described next.

(Risk Factor Extracting Function: Risk Contribution Degree Calculation Function)

The risk contribution degree calculation function 171 is a function for calculating the influence intensity (risk contribution degree) of each biomarker with respect to each illness risk. In the present embodiment, the proportional hazard model (see equation (1)) is used for the calculation of the illness risk, and thus the influence intensity of a specific factor x_{J} can be back calculated with the following equation (3). Here, E_{J}^{(K)} is the influence intensity of the factor x_{J} with respect to the illness risk R_{K} of the illness K.

Influence Intensity Calculation Model with Respect to Illness Risk:

E_{J}^{(K)}=log {R_{K}/R_{K(t)}}×{α_{JK}x_{J}/(Σα_{jK}x_{J}+ε_{K})} (3)

The influence intensity E_{J} of a specific factor x_{J} on the comprehensive health index (health age) can be calculated as the sum of the influence intensity on the individual illness risk as expressed in equation (4). Here, E_{J}^{(CE)} is the influence intensity on the apoplexy risk R_{CE}, E_{J}^{(AC)} is the influence intensity on the coronary arterial abnormality risk R_{AC}, and E_{J}^{(CV)} is the influence intensity on the cardiovascular related illness risk R_{CV}.

Influence Intensity Calculation Model with Respect to Health Age:

E_{J}=E_{J}^{(CE)}+E_{J}^{(AC)}+E_{J}^{(CV)} (4)

The influence intensity of the individual biomarker calculated in the above manner is used for the display of the health profile (see FIG. 7). The contribution degree with respect to the lowering of the evaluation of the health condition (i.e., rise in health age or rise in illness risk) becomes greater for the biomarker of greater influence intensity. Therefore, it is effective to improve the value of the biomarker of great influence intensity to improve health. In the example of FIG. 7, it can be seen that lowering the “systolic blood pressure” is the most effective.

However, the biomarker such as the systolic blood pressure, the blood glucose value, and weight is difficult to be freely controlled by person according to his/her will. Thus, it is not appropriate to present the improvement of the biomarker such as “lower the blood pressure value to xx” as an advice for improving health. The user does not know what kind of action to take in specific to realize the presented target value.

Therefore, in the present embodiment, the factor to be improved is selected from the lifestyle index by the risk factor extracting function described next. The lifestyle index, that is, the body activity and the lifestyle habit can be basically controlled by the will of the person, and thus can be easily connected to a specific improvement activity for the user.

(Risk Factor Extracting Function: Improvable Factor Extracting Function)

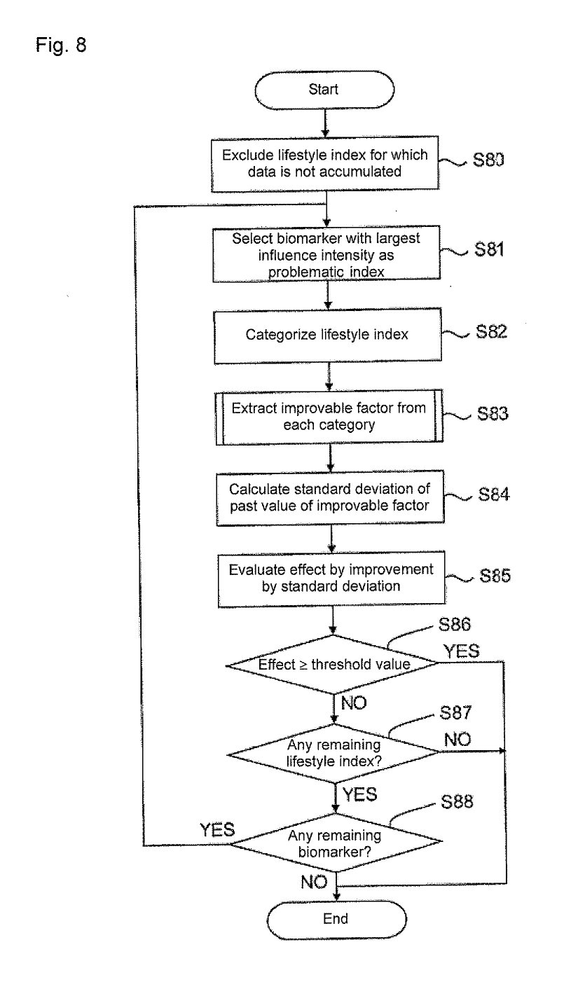

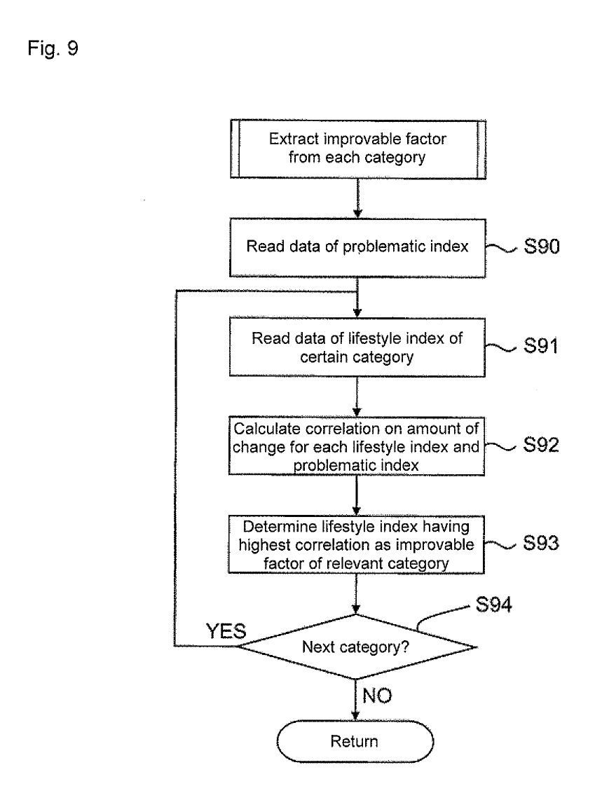

The improvable factor extracting function 172 evaluates which factor is effective to improve in order to improve the health condition of the user, and extracts the factor in which the improvement effect can be expected and that can be improved by the will of the user as an improvable factor. The flow of the extracting process of the improvable factor by the improvable factor extracting function 172 will be described with reference to the flowcharts of FIG. 8 and FIG. 9.

The improvable factor extracting function 172 first examines the type of lifestyle index for which data is accumulated with reference to the user information DB (S80). The lifestyle index for which data is not accumulated is excluded from the candidate of the improvable factor.

The biomarker having the largest cumulative total of the influence intensity (E_{J} in equation (4)) is selected as the problematic index from the biomarker (S81). In the case of the display example at the right side in the upper level of FIG. 7, the “systolic blood pressure” is first selected as the problematic index.

The plurality of lifestyle indices selected in S80 is then classified into three categories of “diet”, “exercise”, and “sleep” (S82), and one improvable factor is extracted from each category (S83). FIG. 9 is a flowchart showing the details of the improvable factor extracting process of S83. The improvable factor extracting function 172 reads the data of the problematic index from the user information DB (S90). The data is read from the user information DB with respect to each lifestyle index belonging to the first category (S91). In S90 and 891, the data in the period necessary for evaluating the correlation of the lifestyle index and the problematic index is read. The data in the previous few weeks is assumed to be read herein. The improvable factor extracting function 172 evaluates the correlation of the amount of change of the lifestyle index and the amount of change of the problematic index (biomarker) (S92). If it is known beforehand that there is a predetermined time delay between the change in the lifestyle index and the change in the problematic index, the correlation may be evaluated in view of such time delay. After calculating the correlation of each lifestyle index belonging to the categories, the improvable factor extracting function 172 determines the lifestyle index having the highest correlation as the improvable factor of the relevant category (S93). After the processes of S91 to S93 are executed for each category (S94), and the improvable factor of each category is determined, the process proceeds to S84 of FIG. 8.

In S84, the improvable factor extracting function 172 calculates the standard deviation a from the past value (may be the same as the data used in the calculation of the correlation) of each improvable factor. The reduction degree of the individual illness risk when all the improvable factors are improved by the standard deviation is then calculated, and all the illness risk reduction degrees are combined (S85). The reduction degree of the illness risk may be calculated based on the correlation obtained in S92 or may be calculated through a method similar to the calculation of the lifestyle improvement effect to be described later.

The processes of S81 to S85 are repeated until the total of the reduction degree of the illness risk reaches a predetermined threshold value (S86; YES), the lifestyle index that can be extracted as the improvable factor does not exist (S87; NO), or the biomarker that can be selected as the problematic index does not exist (S88; NO).

One or a plurality of improvable factors is extracted through the above processes. In the present embodiment, the lifestyle index effective in the improvement of the problematic index and, further, the improvement of the illness risk and the health age can be extracted since the correlation (causal relationship) between the lifestyle index and the problematic index is evaluated using the past data of the user himself/herself accumulated in the user information DB. Furthermore, the improvement proposal regarding various activities and habits in the daily life can be made by extracting the improvable factor by categories, and hence the choices of the user can be extended.

In the present embodiment, the process of S81 of the improvable factor extracting function 172 corresponds to the problematic index selecting means of the present invention, and the processes of S90 to S93 of the improvable factor extracting function 172 corresponds to the improvable factor extracting means of the present invention.

(Risk Factor Extracting Function: Risk Factor Display Function)

The risk factor display function 173 is a function for displaying the improvable factor (lifestyle index) selected by the improvable factor extracting function 172 on the panel 108. The lower level of the screen of FIG. 7 is the display example of the improvable factor. In this example, the “number of continuous walking” and the “continuous walking time” are displayed for the lifestyle index of the exercise category; the “sleeping time” and the “sleeping depth” are displayed for the lifestyle index of the sleep category, and the “alcohol ingestion frequency” is displayed for the lifestyle index of the diet category. The “dinner time” in the diet category is in the non-active state since the data is not accumulated in the user information DB. The “number of cigarettes” is not a factor selected by the improvable factor extracting function 172, but is selected as a default improvable factor as it is known to have great influence on the illness risk.

In the example of FIG. 7, the individual improvable factor is bar displayed. Each part configuring the bar 230 will be described taking the “continuous walking time” by way of example. The vertical axis of the bar 230 represents the value of the lifestyle index, where the value becomes more satisfactory towards the upper side. The color (shading) of the bar 230 shows the variation in the value of the lifestyle index for a certain period (e.g., few weeks) in the past. That is, the portion of darkest color in the bar 230 represents the average μ of a certain period in the past. The width of the bar 230 represents the intensity of the influence the respective improvable factor has on the biomarker (problematic index). That is, the more the improvement effect can be expected, the wider the width of the improvable factor becomes. A black line 231 arranged overlapping the bar 230 shows the immediate measurement value (may be the latest measurement value or average of a plurality of immediate measurement values). A white line 232 on the black line 231 shows the improvement target set by the user. The numerical value “+0.5h” displayed at the upper part of the bar 230 shows the value of the improvement target. A black triangle 233 arranged on the left side of the bar 230 represents the average value in a group of the same age and the same gender as the user.

A second bar 234 arranged on the right side of the bar 230 shows the range that can be set for the improvement target (movable range of the line 232). The position at the upper end of the second bar 234, that is, the upper limit of the value that can be set as the improvement target is set as μ+Nσ (N is an integer greater than or equal to two) using the average μ and the standard deviation σ of the values in the certain period in the past of the improvable factor calculated by the improvable factor extracting function 172. The lower end of the second bar 234, that is, the lower limit of the value that can be set as the improvement target is set at the same value as the immediate measurement value (line 231). Therefore, the setting of an appropriate improvement target that can be realized in a reasonable level for the user can be assisted by determining the upper limit of the improvement target based on the distribution (fluctuation) of the value of the improvable factor of the user himself/herself. Furthermore, the user himself/herself can grasp the improvement amount of the realistic health condition at the current time point by looking at the length of the second bar 234.

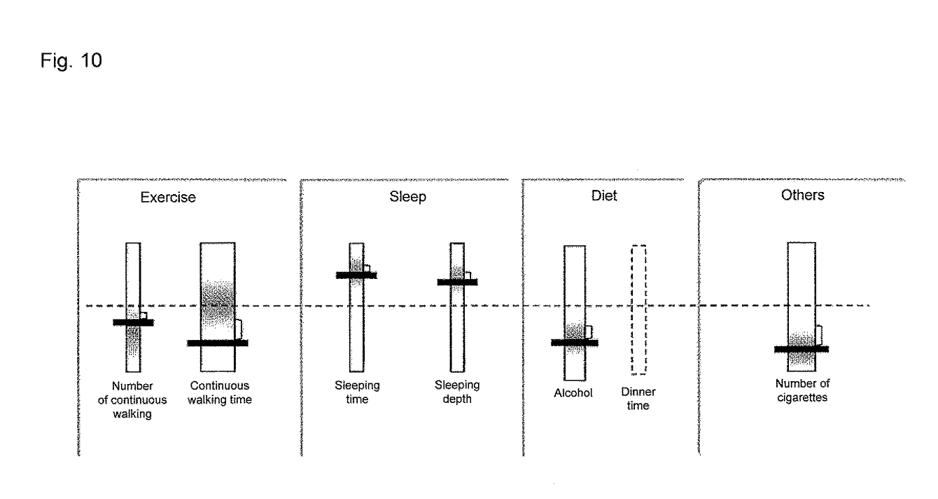

The display example of FIG. 7 is merely an example, and the improvable factor can be displayed in any display format as long as the GUI for setting the improvement target is provided. FIG. 10 shows another display example of the improvable factor. In FIG. 10, the upper and lower positions of the bar are adjusted so that the average value in the group of the same age and the same gender is positioned exactly at the middle of the bar. In this case, the part such as the black triangle 233 of FIG. 7 does not need to be displayed.

(Improvement Plan Assisting Function: Risk Factor Target Setting Function)

The risk factor target setting function 181 is a function for causing the user to set the improvement target with respect to the improvable factor (improvement target selling means). Specifically, as shown in FIG. 7, the improvement target is set by moving the white line 232 on the bar 230 or by directly inputting numerical values.

(Improvement Plan Assisting Function: Lifestyle Improvement Effect Calculation Function)

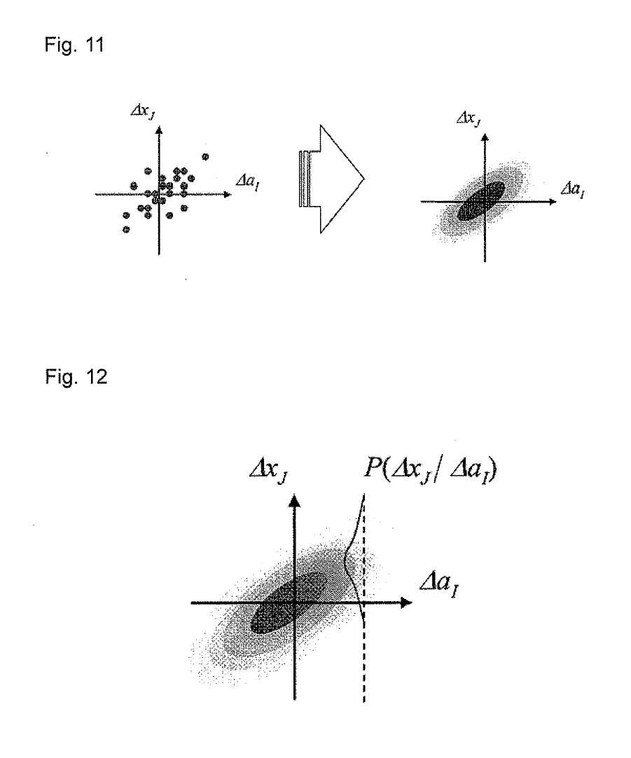

The lifestyle improvement effect calculation function 182 is a function for predicting the improvement effect in the biomarker when the improvement target of the improvable factor is achieved. The method of predicting the improvement effect will now be described with reference to FIG. 11 to FIG. 13.

FIG. 11 schematically shows the method of modeling the influence the change in a certain lifestyle index I has on the value of the biomarker J. The lifestyle improvement effect calculation function 182 first uses the data (measurement value or input value) accumulated in the user information DB to map the relationship of the amount of change Δa_{I} of the lifestyle index I and the amount of change Δx_{J} of the biomarker J as a two-dimensional distribution. A model in which the two-dimensional distribution is approximated with a normal distribution is then generated. If the data of the user himself/herself is not sufficiently accumulated and does not meet a predetermined amount, the lifestyle improvement effect calculation function 182 uses the initial data set prepared in advance in the ROM for the generation of the model. Such data set indicates the statistical value of the group of the same age and the same gender as the user obtained from a large scale follow-up research. Such data set is used if the accumulation of personal data is small, for example, at the beginning of use of the system, and then switched to the personal data at the stage a predetermined amount of personal data is obtained. This enables a reasonable model building of a certain extent from the beginning of use, and the prediction accuracy of the improvement effect can be guaranteed. The model is updated as the personal data is accumulated, so that the prediction accuracy of the improvement effect can be enhanced.

FIG. 12 schematically shows the method of obtaining the improvement effect from the model of FIG. 11. If Δa_{I} is set as the value of the improvement target, the lifestyle improvement effect calculation function 182 obtains the conditional probability distribution P(Δx_{J}|Δa_{I}) from the mode with the target value Δa_{I} as the advance condition. Such conditional probability distribution represents the improvement effect expected on the biomarker J when the lifestyle index I is improved by Δa_{I}.

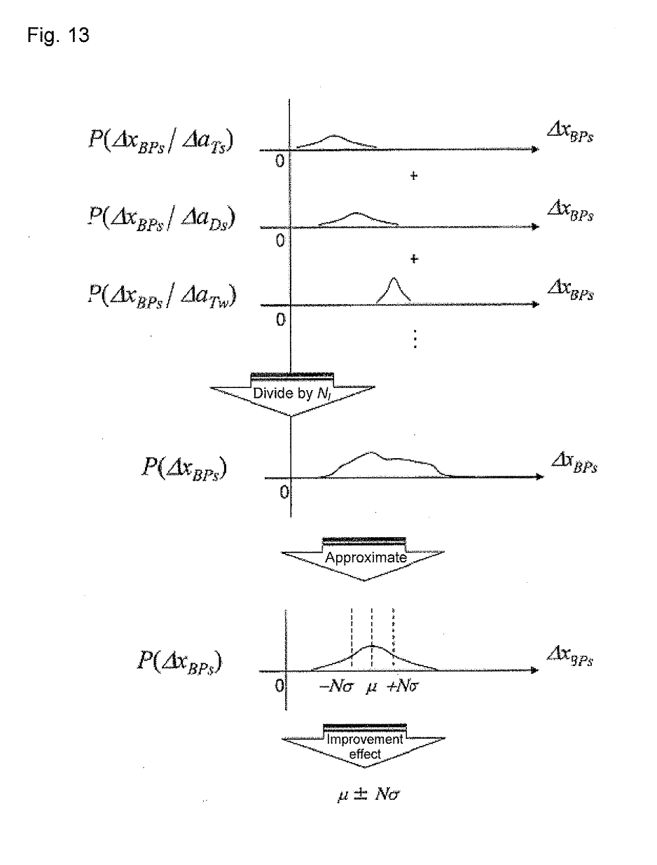

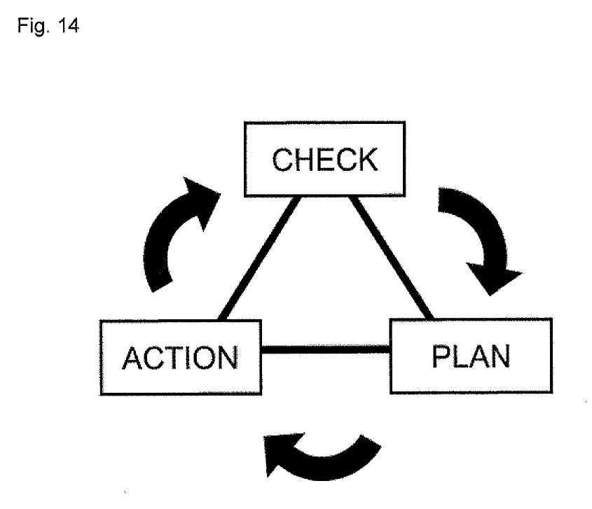

FIG. 13 schematically shows the method of predicting the improvement effect when the improvement target is set to a plurality of lifestyle indices. If the improvement target is given to each lifestyle index Tₛ, Dₛ, T_{w}, . . . , the lifestyle improvement effect calculation function 182 calculates the conditional probability distribution according to the target value of each lifestyle index, similarly as described with FIG. 11 and FIG. 12. The conditional probability distributions are then combined and the combined value is normalized with the parameter N₁ of the lifestyle index. The normalized probability distribution is then approximated as the normal distribution, and the average μ and the standard deviation σ are defined as the improvement effect.

(Improvement Plan Assisting Function: Lifestyle Improvement Effect Display Function)

After the improvement effect μ±Nσ of the biomarker I is obtained, the illness risk evaluating function 162 takes such improvement effect into consideration and calculates each illness risk R_{K}. The health age converting function 163 then uses the illness risk R_{K} to estimate the health age A_{H}. The illness risk and the health age expected when the improvement target is achieved thus can be predicted.

The illness risk and the health age after the improvement obtained in the above manner are displayed on a screen by the lifestyle improvement effect display function 183. In the example of FIG. 7, the health age 203 after the improvement is displayed overlapping the health age distribution at the left on the upper level, and the illness risk distribution 212 after the improvement is displayed in the radar chart at the middle on the upper level.

According to the configuration described above, the improvement effect of the illness risk and the health age is shown in real time by setting the improvement target with respect to the improvable factor. Thus, how the improvement in body activity and lifestyle habit in the daily life influences the comprehensive health condition can be visualized, and simulated. Therefore, very convincing information can be provided to the user, and the motivation with respect to improving health can be maintained and enhanced.

The configuration of the embodiment described above merely illustrates one specific example of the present invention. The scope of the invention is not limited to the embodiments described above, and various modifications can be made within the scope of the technical concept.

### DESCRIPTION OF SYMBOLS

1 comprehensive health condition determining device
2 to 6 measurement device
130 function transition control function
140 initial setting function
141 date and time setting function
142 attribute information setting function
150 biological information measuring function
151 lifestyle habit setting function
152 measurement value communication function
153 measurement value reliability evaluating function
154 measurement value recording function
160 risk estimating function
161 health condition extracting function
162 illness risk evaluating function
163 health age converting function 1
164 health condition display function
165 health age display function
166 health profile display function
170 risk factor extracting function
171 risk contribution degree calculation function
172 improvable factor extracting function
173 risk factor display function
180 improvement plan assisting function
181 risk factor target setting function
182 lifestyle improvement effect calculation function
183 lifestyle improvement effect display function
200 health age distribution
201 health age
202 average value
203 health age after improvement
210 illness risk distribution
211 average illness risk distribution
212 illness risk distribution after improvement
220 influence intensity distribution with respect to coronary arterial abnormality risk
221 influence intensity distribution with respect to cardiovascular related illness risk
222 influence intensity distribution with respect to apoplexy risk
230 bar
231 line showing immediate measurement value
232 line showing improvement target
233 triangle showing average value in group of same age and same gender
234 second bar showing range that can be set for improvement target

1-11. (canceled)

12. A health condition determining device comprising:
a storage means for accumulating data measured or input by an evaluation subject for each index of a plurality of items including a lifestyle index, which is an index related to body activity or lifestyle habit, and a biomarker, which is an index related to physiological state of the body;
an evaluating means for evaluating a health condition of the evaluation subject based on a plurality of indices including the biomarker;
a problematic index selecting means for selecting one or a plurality of biomarkers that lowers the evaluation of the health condition of the evaluation subject from the biomarkers used in the evaluation of the evaluating means as a problematic index;
an improvable factor extracting means for reading data for a predetermined period in the past for the lifestyle index and the problematic index of the evaluation subject from the storage means, and evaluating correlation between amount of change in the lifestyle index and amount of change in the problematic index to extract one or a plurality of lifestyle indices having the highest correlation with the selected problematic index as an improvable factor; and
a display means for displaying the improvable factor along with the evaluation of the health condition of the evaluation subject.

13. The health condition determining device according to claim 12, further comprising:
an improvement target setting means for causing the evaluation subject to set an improvement target with respect to the improvable factor; and
an improvement effect calculation means for predicting the improvement effect in the problematic index when the improvement target is achieved after the improvement target is set by the improvement target setting means; wherein
the evaluating means evaluates the health condition after the improvement by taking the improvement effect of the problematic index into consideration, and the display means further displays the improvement target of the improvable factor and the evaluation of the health condition after the improvement.

14. The health condition determining device according to claim 13, wherein the improvement target setting means sets an upper limit of a value that can be set as the improvement target based on a distribution of values of the improvable factors accumulated in the storage means.

15. The health condition determining device according to claim 13, wherein the improvement effect calculation means models influence of the value of the improvable factor and change thereof on the value of the problematic index based on the past data of the improvable factor and the problematic index accumulated in the storage means, and calculates the improvement effect of the problematic index using the model.

16. The health condition determining device according to claim 15, wherein if the past data of the evaluation subject accumulated in the storage means does not meet a predetermined amount, the improvement effect calculation means also uses data prepared in advance in the storage means to generate the model.

17. The health condition determining device according to claim 12, wherein the display means further displays the evaluation of the health condition and an average value of the improvable factor in a person of the same age and same gender as the evaluation subject.

18. The health condition determining device according to claim 12, wherein if there are plural the improvable factors, the display means further displays intensity of the influence of the respective improvable factors on the problematic index.

19. A health condition determining system comprising:
the health condition determining device according to claim 12: and
one or a plurality of measurement devices for measuring the index from the evaluation subject.

20. A health condition determining method in which a computer executes the steps of:
accumulating data measured or input to a storage means by an evaluation subject for each index of a plurality of items including a lifestyle index, which is an index related to body activity or lifestyle habit, and a biomarker, which is an index related to physiological state of the body;
evaluating a health condition of the evaluation subject based on a plurality of indices including the biomarker;
selecting one or a plurality of biomarkers that lowers the evaluation of the health condition of the evaluation subject from the biomarkers used in the evaluation in the evaluating step as a problematic index;
reading data for a predetermined period in the past for the lifestyle index and the problematic index of the evaluation subject from the storage means, and evaluating correlation between amount of change in the lifestyle index and amount of change in the problematic index to extract one or a plurality of lifestyle indices having the highest correlation with the selected problematic index; and
displaying the improvable factor along with the evaluation of the health condition of the evaluation subject.

21. A program for causing a computer to execute each step in the health condition determining method according to claim 20.

22. A computer readable recording medium recorded with the program according to claim 21.

23. A health condition determining system comprising:
the health condition determining device according to claim 13: and
one or a plurality of measurement devices for measuring the index from the evaluation subject.

24. A health condition determining system comprising:
the health condition determining device according to claim 14: and
one or a plurality of measurement devices for measuring the index from the evaluation subject.

25. A health condition determining system comprising:
the health condition determining device according to claim 15: and
one or a plurality of measurement devices for measuring the index from the evaluation subject.

26. A health condition determining system comprising:
the health condition determining device according to claim 16: and
one or a plurality of measurement devices for measuring the index from the evaluation subject.

27. A health condition determining system comprising:
the health condition determining device according to claim 17: and
one or a plurality of measurement devices for measuring the index from the evaluation subject.

28. A health condition determining system comprising:
the health condition determining device according to claim 18: and
one or a plurality of measurement devices for measuring the index from the evaluation subject.
